(19) United States
(12) Patent Application Publication
(10) Pub. No.: US 2014/0206632 A1
(43) Pub. Date: Jul. 24, 2014

(54) Endothelin in the Diagnosis of Cardiac Disease

(71) Applicant: Singulex, Inc., Alameda, CA (US)

(72) Inventors: John Todd, Lafayette, CA (US); Joel Estis, Albany, CA (US); Victoria Torres, Concord, CA (US)

(73) Assignee: Singulex, Inc., Alameda, CA (US)

(21) Appl. No.: 14/161,420

(22) Filed: Jan. 22, 2014

Related U.S. Application Data
(60) Provisional application No. 61/755,154, filed on Jan. 22, 2013.

(51) Int. Cl. G01N 33/543

(52) U.S. Cl. CPC G01N 33/54326
USPC 514/26; 436/501; 514/165; 514/56; 514/91; 514/211.07

(57) ABSTRACT

A method for determining the risk, severity or progression of cardiovascular disease, such as cardiac heart failure. A method for determining the likelihood of admission to the hospital for cardiac heart failure. The methods include determining the concentration of ET-1 and the concentration of one or more of biomarkers selected from the group consisting of cardiac troponin (e.g., cTnI, cTnT), VEGF, BNP, NT-proBNP, and IL-6 in a blood, serum or plasma sample from the patient.

### BACKGROUND

Following hospitalization for heart failure, hospital readmission is a common, costly, and often preventable outcome that is increasingly being viewed by payers and policymakers as an indicator of hospital-level quality and efficiency of care. Up to one-third of heart failure patients are readmitted to the hospital within 30 days of being discharged. Research shows that close follow-up with patients after they have been discharged can significantly reduce readmission rates. In addition, studies point to the success of various quality improvement interventions—such as pairing patients with peer advisors after discharge, disease management programs administered by home health nurses, and enrollment in cardiac rehabilitation programs—that reduce the risk of readmission and improve outcomes for patients with heart failure.

Implementing interventions to reduce readmission after heart failure will require an understanding of the patient characteristics associated with readmission. Knowledge of relevant patient characteristics will help physicians stratify heart failure patients according to risk of readmission and assist with tailoring discharge plans. In addition, any attempt to meaningfully compare rates of readmission across hospitals will require the ability to adequately and appropriately risk-adjust for differences in patient characteristics. Risk adjustment will help to ensure that measures designed to identify outlier hospitals with respect to readmission rates are doing so based on differences in the quality of heart failure-related care rather than differences in the sociodemographic and clinical characteristics of the patient population.

Accordingly, the inventors have identified a need in the art for the ability to accurately predict whether a heart failure patient will require admission to the hospital so that supporting interventions to reduce the likelihood of readmission can be implemented, which will reduce costs and improve the quality and outcomes of care for heart failure patients.

### SUMMARY

In one embodiment, the disclosure is directed to a method for determining the risk or severity of cardiac disease. The method includes determining the concentration of Endothelin 1 (ET-1) and at least one of cardiac troponin, Vascular Endothelial Growth Factor (VEGF), Tumor Necrosis Factor alpha TNF-α, Brain Natriuretic Peptide (BNP), proBNP, N-terminal proBNP (NT-proBNP), Interleukin-6 (IL-6), in a blood, serum or plasma sample from a patient, and using the concentrations to predict the risk or severity of cardiac disease. In aspects of this and other embodiments, the cardiac disease may be cardiac heart failure (CHF), and the patient may have been hospitalized for heart failure. Also, in one aspect, the concentrations of (ET-1) and at least one of cardiac troponin, VEGF, IL-6, BNP, proBNP, NT-proBNP, and TNF-α in a blood sample from a patient are compared to the concentrations in a population of normal healthy control subjects. In another aspect, the concentrations of ET-1 and at least one of cardiac troponin, VEGF, IL-6, BNP, proBNP, NT-proBNP, and TNF-α in a blood sample from a patient are compared to the concentrations in a population of patients that have been admitted to the hospital for cardiac heart failure. Still further, the risk or severity of cardiac disease may be the likelihood of admission to a hospital following the diagnosis of cardiac heart failure.

In another embodiment, the disclosure is directed to a method for determining the likelihood of hospital admission for heart failure. The method includes determining the concentration of ET-1 and the concentration of one or more of biomarkers selected from the group consisting of cardiac troponin, VEGF, BNP, proBNP, NT-proBNP, TNF-α and IL-6 in a blood, serum or plasma sample from the patient. The concentration of ET-1 in the sample is compared to the concentration ET-1 in blood, serum or plasma samples from a population of normal healthy control subjects. The concentration of the one or more biomarkers in the sample is compared to the concentration of the at least one or more biomarkers in samples from a population of normal healthy control subjects. The likelihood of hospital admission is determined when at least one of the concentration of ET-1 and the concentration of cTnI in the patient sample is elevated above the normal healthy concentrations. In various aspects of this embodiment, the comparing may include the identification of a CHF score. The score may include an odds ratio related to hospital admission following a diagnosis of heart failure or previous hospital admission for heart failure. The odds ratio may be determined by predicting the presence of CHF by the coded risk categories (e.g., age, gender, and/or hospital admission) in a logistic regression model.

In a further embodiment, the disclosure is directed to a method of determining the likelihood of a hospital admission for heart failure in a patient diagnosed with heart failure. The method includes determining the concentration of ET-1 and the concentration of one or more of biomarkers selected from the group consisting of cTnI, cTnI, VEGF, BNP, proBNP, NT-proBNP, TNF-α and IL-6 in a blood, serum or plasma sample from the patient; determining an odds ratio for ET-1 and the one or more biomarkers for the likelihood of hospital admission for the patient, wherein the odds ratio includes a cut-off value representing the concentration of ET-1 and the one or more biomarkers in a population of heart failure patients that have been admitted to the hospital, and comparing the concentration of ET-1 and the one or more biomarkers in the sample to the cut-off value, thereby determining the likelihood of hospital admission for the patient. In various aspects of this embodiment, the odds ratio may be adjusted for age and sex.

In yet another embodiment, the disclosure is directed to a method of treating CHF. The method includes determining that a patient is suffering from CHF using a method according to the disclosure and administering a CHF treatment to the patient.

Still further, the disclosure is directed to a method for preventing a hospital admission for CHF, the method comprising determining the likelihood of a hospital admission for CHF according the method the disclosure and administering a treatment to the patient for prolonging hospital admission.

### BRIEF DESCRIPTION OF THE DRAWINGS

FIG. 1 is a schematic diagram of Endothelin-1 (ET-1) and its degradation fragments.

FIG. 2 shows the result of assays for ET-1 on normal healthy subjects and subjects suffering from heart failure.

FIG. 3 shows the results of assays with antibody pairs for detecting ET-1.

FIG. 4 shows the results of assays conducted for optimizing the detection of ET-1 using antibodies MA10818 and MAB3440.

FIG. 5 shows the results of assays conducted for optimizing the detection of ET-1 using antibodies MA3440 and MA3005.

FIG. 6 shows the results of experiments for optimizing salt and detergent concentrations for assays for detecting ET-1.

FIG. 7 is a histogram showing the concentration of ET-1 in a population of healthy subjects.

FIG. 8 shows the result of the Singulex ET-1 assay on normal healthy subjects and subjects suffering from heart failure.

FIG. 9 is a survival curve reflecting time to hospitalization for baseline quarties using the Singulex ET-1 assay.

FIG. 10 is a survival curve reflecting time to hospitalization for baseline tertiles using the Singulex cTnI assay.

FIG. 11 is a survival curve reflecting time to hospitalization for baseline quartiles using CT-proET-1 assay.

FIG. 12 is a survival curve reflecting time to hospitalization in a 1300 day study by count of elevated baseline biomarkers Sgx ET-1, cTnI and VEGF.

FIG. 13 is a survival curve reflecting time to hospitalization in a 30 day study by count of elevated baseline biomarkers Sgx ET-1, cTnI and VEGF.

FIG. 14 is a survival curve reflecting time to hospitalization in a 90 day study by count of elevated baseline biomarkers Sgx ET-1, cTnI and VEGF.

### DESCRIPTION

All publications, patent applications, patents and other references mentioned herein, if not otherwise indicated, are explicitly incorporated by reference.

Stratifying heart failure patients according to risk of hospital admission or readmission will help physicians tailor discharge plans and help to prevent admission or readmission. The risk of admission or readmission for HF can be determined by analysing a heart failure patient's blood for the concentration of Endothelin-1 and one or more of the following: Vascular Endothelial Growth Factor (VEGF), Tumour Necrosis Factor alpha (TNF-α), cardiac troponin (e.g., cardiac troponin I (cTnI), cardiac troponin T (cTnT)), brain natriuretic peptide (BNP), proBNP, N-terminal proBNP (NT-proBNP), and Interleukin-6 (IL-6). Analysis may be completed at heart failure diagnosis or hospital discharge, or some point after diagnosis or discharge.

Accordingly, one aspect the disclosure is directed to a method for determining the likelihood of hospital admission or readmission for a patient that has been diagnosed with heart failure or that has been previously hospitalized for heart failure. The method includes determining the concentration of ET-1 and one or more second biomarkers selected from cardiac troponin, cTnI, cTnT, BNP, proBNP, NT-proBNP, IL-6, VEGF, and TNF-α in a blood sample from the patient. The concentration of ET-1 in the sample is compared to the concentration of ET-1 in blood samples from a population of normal healthy control subjects or a population of patients that have been admitted to the hospital for heart failure. The concentration of the one or more second biomarkers in the sample is compared to the concentration of the one or more biomarkers in blood samples from the healthy population or the hospitalized population. The likelihood of hospital admission or readmission is increased when at least one of the concentrations of ET-1 and the second biomarker(s) in the patient sample is elevated above the normal healthy concentrations or about a cut-off value associated with the concentration in the hospitalized population. The method may further include determining an odds ratio for the likelihood of hospital admission or admission for heart failure.

In another aspect, the disclosure is directed to a method for predicting the likelihood of heart failure and diagnosing heart failure. The method includes determining the concentration of ET-1 in combination with one or more of cardiac troponin, VEGF, IL-6, BNP, proBNP, NT-proBNP and TNF-α. Concentrations of ET-1, cardiac troponin, VEGF, IL-6, BNP, proBNP, NT-proBNP and/or TNF-α are compared to the concentrations of these markers in a population of normal healthy control subjects. The likelihood of heart failure or the diagnosis of heart failure can be determined by comparing the levels of each of biomarkers to the normal levels.

Unless otherwise defined, the technical and scientific terms used herein have the same meaning as commonly understood by one of ordinary skill in the art. Expansion and clarification of some terms are provided herein.

As used herein, the term “subject” refers to a human subject. Typically, the terms “subject” and “patient” are used interchangeably.

As used herein, the term “sample” is taken broadly to include any sample suitable for the methods described herein. Typically, the sample is a biological sample such as, for example, a biological fluid. Such fluids can include, without limitation, bronchoalveolar lavage fluid (BAL), blood, serum, plasma, urine, nasal swab, cerebrospinal fluid, pleural fluid, synovial fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the target particle of interest. Other similar specimens such as cell or tissue culture or culture broth are also of interest. In some embodiments, the sample is a blood sample such as whole blood, plasma, or serum samples. Samples can be frozen and stored for later analysis. In addition, samples may require preparation or processing (e.g., dilution) prior to processing.

As used herein, the terms “healthy volunteer average concentrations” or “population of normal healthy control subjects” refer to the average concentration of the various biomarkers described herein for at least two subjects who do not have cardiovascular disease, such as CHF (i.e., “healthy patients”). Preferably, average concentration values are calculated from biomarker concentrations measured in larger groups of healthy volunteers (HVs). Healthy volunteer average concentrations are considered herein, but one of skill in the art may also measure biomarker concentrations in one or more populations of subjects lacking cardiovascular disease by utilizing an apparatus capable of sensitively measuring the concentrations of biomarkers described herein and calculating the average values for each biomarker in such HV populations.

As used herein, the term “substantially the same as” refers to ± about 25%, ± about 20%, ± about 15%, ± about 10%, ± about 5%, ± about 3%, ± about 2%, or ± about 1% of the healthy volunteer average concentrations of a biomarker. In some aspects “substantially the same as” refers to ± about 20%, ± about 15%, ± about 10%, or ± about 5%, ± about 3%, ± about 2%, or ± about 1% of the healthy volunteer average concentrations of a biomarker. In some aspects “substantially the same as” refers to ± about 15%, ± about 10%, ± about 5%, ± about 3%, ± about 2%, or ± about 1% of the healthy volunteer average concentrations of a biomarker. In some aspects “substantially the same as” refers to ± about 10%, ± about 5%, ± about 3%, ± about 2%, or ± about 1% of the healthy volunteer average concentrations of a biomarker. In some aspects “substantially the same as” refers to ± about 5%, ± about 3%, ± about 2%, or ± about 1% of the healthy volunteer average concentrations of a biomarker. In some aspects “substantially the same as” refers to ± about 3%, ± about 2%, or ± about 1% of the healthy volunteer average concentrations of a biomarker. In some aspects “substantially the same as” refers to ± about 2%, or ± about 1% of the healthy volunteer average concentrations of a biomarker. In some aspects “substantially the same as” refers to ± about 1% of the healthy volunteer average concentrations of a biomarker.

As used herein, the term “CV” refers to the coefficient of variance.

As used herein, the term “average CV” refers to average of the coefficient of variance obtained for all samples tested in triplicate.

As used herein, the term “LoD” refers to the limit of detection, defined as 2 standard deviations above the zero calibrator.

As used herein, the term “LLoQ” refers to the lower limit of quantification, defined from data generated off of a standard curve. Specifically, the back interpolated values of standards in triplicate provide CVs<20% and a bias<20% of the expected values.

The terms “diagnosis” and “diagnostics” also encompass the terms “prognosis” and “prognostics”, respectively, as well as the applications of such procedures over two or more time points to monitor the diagnosis and/or prognosis over time and statistical modeling based thereupon. Furthermore the term diagnosis includes: a. prediction (determining if a patient will likely develop a cardiovascular disease), b. prognosis (predicting whether a patient will likely have a better or worse outcome at a pre-selected time in the future), c. therapy selection, d. therapeutic drug monitoring, and e. relapse monitoring.

The terms “admission” or “readmission” are used interchangeably to describe a patient's admission to the hospital following a diagnosis of heart failure. Since not all HF patients are admitted to the hospital upon an initial diagnosis, “admission” refers to patients that have not previously been admitted as well as patients that have been readmitted following a prior admission for heart failure. For the purposes here, readmission and admission should be considered synonymous, regardless of a previous admission.

The term “score” or “scoring” refer to calculating a cut-off value, probability or likelihood for a parameter in a sample. In one example, values closer to 1.0 are used to represent the likelihood that a sample is from a patient suffering from a condition, such as cardiac disease (i.e., CHF), values closer to 0.0 represent the likelihood that the patient does not have a condition. The logistic regression classification method may be used to combine a panel of biomarkers to calculate the probability score between, for example, 0 and 1 for each sample.

The term “coefficients” refers to the weight assigned to each protein used in a logistic regression equation to score a sample.

The term “condition” as used herein refers generally to a disease, event, or change in health status.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, a specificity, positive predictive values (PPV), or negative predictive values (NPV), or as a likelihood or an odds ratio, among other measures.

The term “biomarker” as used herein refers to a polypeptide in a biological sample from a subject. A biomarker protein includes not only the polypeptide itself, but also minor variations thereof, including for example one or more amino acid substitutions or modifications such as glycosylation or phosphorylation. Biomarker levels may change due to treatment or progression of the disease. The changes in biomarker levels may be measured in accordance with the present disclosure. Changes in biomarker levels may be used to monitor the progression of disease or therapy.

The terms “panel” or “biomarker panel” as used herein refers to a plurality of biomarkers, for example, 1, 2, 3 or more biomarkers. In certain embodiments, the levels of the proteins in the panels can be correlated with the existence of a condition in a subject.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage, and the like). Likewise, “providing” may refer to the act of indirectly obtaining the biological sample. For example, providing may refer to the act of a laboratory receiving the sample from the party that directly obtained the sample, or to the act of obtaining the sample from an archive.

The term “sensitivity” refers to a characteristic of a diagnostic test relating to the probability that a patient with the disease will have a positive test result. This is derived from the number of patients with the disease who have a positive test result (true positive) divided by the total number of patients with the disease, including those with true positive results and those patients with the disease who have a negative result, i.e. false negative.

The term “specificity” refers to a characteristic of a diagnostic test relating the probability that a patient without the disease will have a negative test result. This is derived from the number of patients without the disease who have a negative test result (true negative) divided by all patients without the disease, including those with a true negative result and those patients without the disease who have a positive test result, e.g. false positive. While the sensitivity, specificity, true or false positive rate, and true or false negative rate of a test provide an indication of a test's performance, e.g. relative to other tests, to make a clinical decision for an individual patient based on the test's result, the clinician requires performance parameters of the test with respect to a given population.

Cardiovascular disease includes, but is not limited to, congestive heart failure (CHF), high blood pressure, arrhythmias, atherosclerosis, cholesterol, Wolff-Parkinson-White Syndrome, long QT syndrome, angina pectoris, tachycardia, bradycardia, atrial fibrillation, ventricular fibrillation, congestive heart failure, myocardial ischemia, myocardial infarction, cardiac tamponade, myocarditis, pericarditis, arrhythmogenic right ventricular dysplasia, hypertrophic cardiomyopathy, Williams syndrome, heart valve diseases, endocarditis, bacterial, pulmonary atresia, aortic valve stenosis, Raynaud's disease, cholesterol embolism, Wallenberg syndrome, Hippel-Lindau disease, and telangiectasis.

Heart failure (HF), often called congestive heart failure (CHF) or congestive cardiac failure (CCF), occurs when the heart is unable to provide sufficient pump action to distribute blood flow to meet the needs of the body. Heart failure can cause a number of symptoms including shortness of breath, leg swelling, and exercise intolerance. The condition is diagnosed with echocardiography and blood tests. Treatment commonly consists of lifestyle measures such as smoking cessation, light exercise including breathing protocols, decreased salt intake and other dietary changes, and medications. Sometimes it is treated with implanted devices (pacemakers or ventricular assist devices) and occasionally a heart transplant.

Common causes of heart failure include myocardial infarction and other forms of ischemic heart disease, hypertension, valvular heart disease, and cardiomyopathy. The term heart failure is sometimes incorrectly used for other cardiac-related illnesses, such as myocardial infarction (heart attack) or cardiac arrest, which can cause heart failure but are not equivalent to heart failure.

The New York Heart Association (NYHA) Functional Classification provides a simple way of classifying the extent of heart failure. It places patients in one of four categories based on how much they are limited during physical activity:

| NYHA | |
|---|---|
| Class | Symptoms |
| I | Cardiac disease, but no symptoms and no limitation in |
| | ordinary physical activity, e.g., shortness of breath |
| | when walking, climbing stairs etc. |
| II | Mild symptoms (mild shortness of breath and/or angina) |
| | and slight limitation during ordinary activity. |
| III | Marked limitation in activity due to symptoms, even |
| | during less-than-ordinary activity, e.g. walking short |
| | distances (20-100 m). Comfortable only at rest. |
| IV | Severe limitations. Experiences symptoms even while at |
| | rest. Mostly bedbound patients. |

The methods and compositions of the disclosure thus include methods and compositions for the highly sensitive detection and quantitation of ET-1, VEGF, IL-6, and TNF-α, BNP, proBNP, NT-proBNP, IL-6, and cardiac troponin (e.g., cTnI, cTnT), and compositions and methods for diagnosis, prognosis, and/or determination of treatment based on such highly sensitive detection and quantitation. In one particular embodiment, for example, the probability of hospital admission following diagnosis for heart failure can be determined and used in the treatment of the patient to prevent or postpone future admissions or readmissions.

Endothelin-1

Endothelin-1 (ET-1) is a key mediator of vascular tone and renal homeostasis through antagonistic vasoactive effects. ET-1 is a vasoconstrictor, but it also induces the production of the potent vasodilator, nitric oxide (NO). Consequently, dysfunctions of ET-1 signalling are associated with cardiovascular, renal and respiratory diseases, such as high blood pressure, atherosclerosis, systemic and pulmonary hypertension.

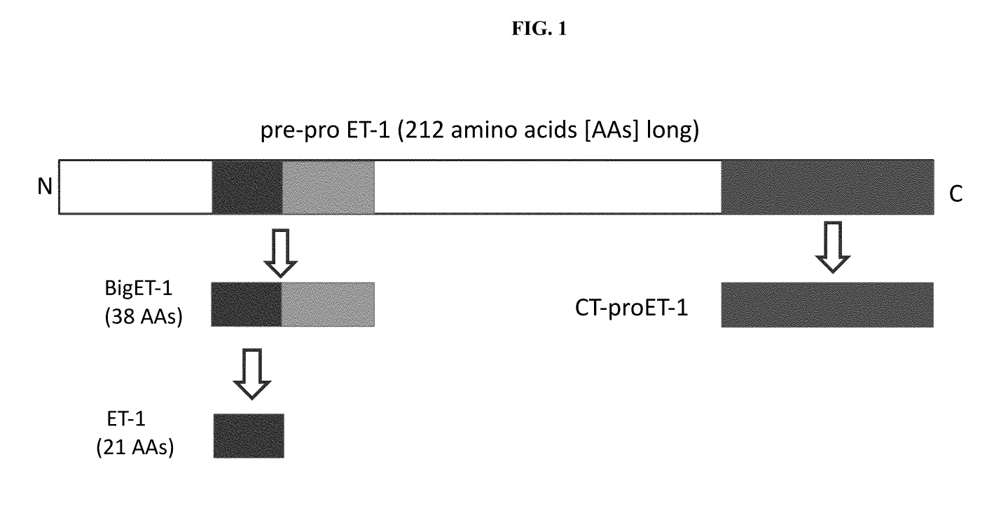

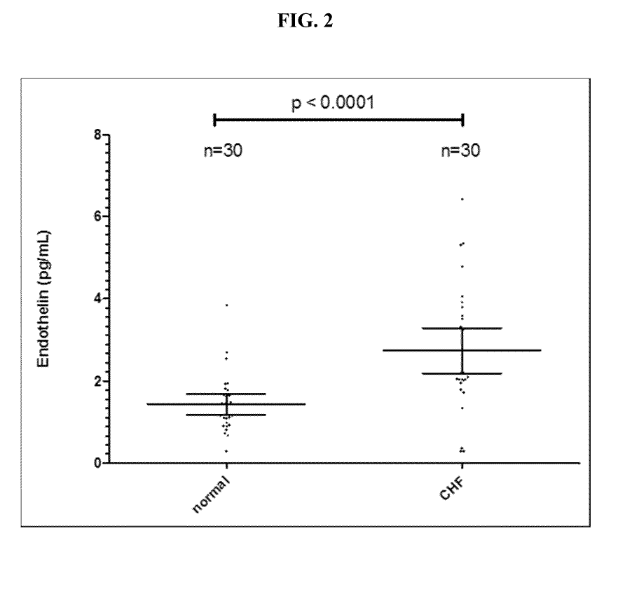

Endothelin-1 is expressed by endothelial cells as a precursor peptide (proET-1) that is first cleaved to bigET-1 and then to the mature 21-amino acid peptide. (See FIG. 1.) The half life of ET-1 in plasma is about 1-2 minutes. Its concentration in healthy humans is 1-2 pg/mL. A radio immunoassay for ET-1 was first described in 1989. Since then, ELISA assay kits have been developed. A commercially available ELISA kit for the detection of mature ET-1 is available, for example, from R&D Systems. This kit has a Lower Limit of Quantitation of approximately 1-2 pg/mL. In addition, an assay kit is available for the detection of the C-terminal portion of ET-1 (CT-proET-1). Results of assays for ET-1 in healthy (n=30) and diseased (n=30) subjects using the R&D Systems Quantikine kit to measure the 21 amino acid ET-1 molecule are shown in FIG. 2.

For the purposes of this disclosure, ET-1 refers to all forms of endothelin-1 unless otherwise noted. Therefore ET-1 generally refers to the various forms of ET-1, including, but not limited to, bigET-1, proET-1 and CT-proET-1.

Cardiac Troponin

When the two unique forms of cardiac troponin, cardiac troponin I (cTnI) and cardiac troponin T (cTnT) are released into the blood from cardiac muscle, several species of each may exist in the blood. These include various complexes of the two forms, with each other and/or with cardiac troponin C (cTnC). In addition, the two forms are subject to virtually immediate proteolytic degradation, resulting in a variety of fragments. Also, various phosphorylated and oxidized forms of the troponins may exist in the blood. See, e.g., U.S. Pat. No. 6,991,907, which is incorporated by reference herein in its entirety. Unless otherwise specified, “cardiac troponin,” as used herein, encompasses all forms of cardiac troponin.

In some embodiments, the disclosure provides methods and compositions for the detection and/or determination of concentration of total cardiac troponin, i.e., the sum of all or a substantial portion of the cardiac troponin in a sample, e.g., blood, serum or plasma sample, whether it is free, complexed, a proteolytic fragment, phosphorylated, oxidized, or otherwise modified. In some embodiments, the cardiac troponin is cTnI, in others it is cTnT, and in still other embodiments, the cardiac troponin is cTnI and cTnT. It will be appreciated that an absolute total measurement need not be achieved, as long as a consistent proportion of the total is determined, which can be compared to standard values. It will also be appreciated that if a form of troponin is a minor constituent of the total, absence or low levels of detection of that form will not appreciably affect measures of total troponin. Thus, as used herein, “total cardiac troponin” refers to a measurement that is intended to measure all or substantially all forms of a particular cardiac troponin, e.g., all cTnI, or all cTnT, in a sample, where the sample-to-sample consistency is such that clinically relevant conclusions may be drawn from comparisons of samples to standards or comparison of one sample to another.

Interleukin 6 (IL-6)

Interleukin-6 (IL-6) is a pro-inflammatory cytokine secreted by T cells and macrophages to stimulate immune response to trauma, especially burns or other tissue damage leading to inflammation. IL-6 is also secreted by macrophages in response to specific microbial molecules, referred to as pathogen associated molecular patterns (PAMPs), which trigger the innate immune response and initiate inflammatory cytokine production. IL-6 is one of the most important mediators of fever and of the acute phase response. IL-6 is also called a “myokine,” a cytokine produced from muscle, and is elevated in response to muscle contraction. Additionally, osteoblasts secrete IL-6 to stimulate osteoclast formation.

IL-6-related disorders include but are not limited to sepsis, peripheral arterial disease, and chronic obstructive pulmonary disease. Interleukin-6 mediated inflammation is also the common causative factor and therapeutic target for atherosclerotic vascular disease and age-related disorders including osteoporosis and type 2 diabetes. In addition, IL-6 can be measured in combination with other cytokines, for example TNFα to diagnose additional diseases such as septic shock. IL-6 has therapeutic potential as a drug target which would result in an anti-inflammatory and inhibition of the acute phase response. In terms of host response to a foreign pathogen, IL-6 has been shown, in mice, to be required for resistance against the bacterium Streptococcus pneumoniae. Inhibitors of IL-6 (including estrogen) are used to treat postmenopausal osteoporosis. There is also therapeutic potential for cancer, as IL-6 is essential for hybridoma growth and is found in many supplemental cloning media such as briclone.

The determination of IL-6 using highly sensitive single molecule counting assays is described, for example, in U.S. Pat. No. 8,450,069 and U.S. Patent Application Publication No. US20100112727, which are incorporated reference herein in their entirety.

VEGF

Vascular endothelial growth factor-A (VEGF-A), commonly known as VEGF, is a member of a family of secreted glycoproteins that promote endothelial cell growth, survival, migration, and vascular permeability, all of which contribute to angiogenesis. The binding of VEGF to its receptor triggers the activation of a cell signaling pathway that is critical for the growth of blood vessels from pre-existing vasculature. VEGF is implicated in a variety of diseases including cardiac conditions, cancer, age-related macular degeneration, diabetic retinopathy and rheumatoid arthritis. As such, it is an attractive candidate for the development of therapies to these diseases.

The determination of VEGF using highly sensitive single molecule counting assays is described, for example, in U.S. Pat. No. 8,450,069.

TNF-α

Tumor necrosis factor (TNFα) is an adipokine involved in systemic inflammation and is a member of a group of cytokines that stimulate the acute phase reaction. It is produced chiefly by activated macrophages (Ml), although it can be produced by many other cell types such as CD4+ lymphocytes, NK cells and neurons.

The determination using highly sensitive single molecule counting assays is described, for example, in U.S. Pat. No. 8,450,069.

BNP

Brain natriuretic peptide (BNP), now known as B-type natriuretic peptide or Ventricular Natriuretic Peptide (still BNP), is a 32-amino acid polypeptide secreted by the ventricles of the heart in response to excessive stretching of heart muscle cells (cardiomyocytes). The release of BNP is modulated by calcium ions. BNP is secreted along with a 76-amino acid N-terminal fragment (NT-proBNP) that is biologically inactive. BNP is produced from a preprohormone precursor. Removal of a 26-amino acid signal peptide gives rise to a 108-amino acid proBNP. Several commercial assays are available for the detection of BNP, proBNP and NT-proBNP. In addition, determination of the various forms of BNP using highly sensitive single molecule counting assays is described, for example, in U.S. Pat. No. 8,450,069.

Determination of Diagnosis, Prognosis, or Method of Treatment

Normal values, threshold values, rates of change, ratios of values, odds ratios, hazard ratios, and other useful diagnostic and prognostic indicators may be established by methods well-known in the art. For example, these values may be determined by comparing samples from a CHF population and a control population, where the CHF population exhibits the biological state for which diagnosis, prognosis, or method of treatment is desired, and the control population does not exhibit the biological state. In some embodiments, a longitudinal study may be done, e.g., the CHF population may be a subset of the control population that, over time, exhibits the biological state. It will be appreciated that data from a plurality of studies may be used to determine a consensus value or range of values for normal, and for prognostic or diagnostic levels.

In developing diagnostic or prognostic test, data for one or more potential markers may be obtained from a group of subjects. The group of subjects is divided into at least two sets, and preferably the first set and the second set each have an approximately equal number of subjects. The first set includes subjects who have been confirmed as having a disease or, more generally, being in a first condition state. For example, this first set of patients may be those that have recently had a disease incidence, or may be those having a specific type of disease, such as CHF. The confirmation of the condition state may be made through a more rigorous and/or expensive testing such as MRI or CT. Hereinafter, subjects in this first set will be referred to as “diseased”. The second set of subjects is simply those who do not fall within the first set. Subjects in this second set may be “non-diseased”, that is, healthy or normal subjects. Alternatively, subjects in this second set may be selected to exhibit one symptom or a constellation of symptoms that mimic those symptoms exhibited by the “diseased” subjects; for example patients that have been previously diagnosed or hospitalized for cardiac disease. In still another alternative, this second set may represent those at a different time point from disease incidence. Preferably, data for the same set of markers is available for each patient. This set of markers may include all candidate markers which may be suspected as being relevant to the detection of a particular disease or condition. Actual known relevance is not required. Embodiments of the compositions, methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

In various embodiments of the methods described herein, the sample can be a single sample from the subject. In some embodiments, the sample can be a series of samples taken at various points in time so that changes in concentration over time of a biomarker related to cardiac disease, including heart failure, can be identified and interpreted. The samples can be taken in over the course of hours, days, weeks, months, and years. The samples can be taken at any regular or irregular interval based on the detected concentration(s) of biomarker related to cardiac disease, including heart failure, and/or the change in the concentration(s) of biomarker related to cardiac disease, including heart failure, in the one or more samples over time.

In embodiments that track patient data and samples over time, such information can be taken from any known clinical study or database that maintains such patient samples and/or patient history.

Some embodiments of the disclosure include comparing said concentration or series of concentrations to a normal value for said concentration, comparing said concentration or series of concentrations to a predetermined threshold level, comparing said concentration or series of concentrations to a baseline value, or determining a rate of change of concentration for said series of concentrations.

Some embodiments of the disclosure include comparing said concentration of one or more biomarkers in the sample with a predetermined threshold concentration, and determining a diagnosis, prognosis, or method of treatment if the sample concentration is greater than the threshold level. The threshold concentration can be determined by, e.g., determining the 99th percentile concentration of the biomarker in a group of individuals, and setting said threshold concentration at said 99th percentile concentration.

In another aspect, the disclosure relates to a method for diagnosing CHF in a subject comprising determining an amount of ET-1 and cTnI in a blood, serum, or plasma sample from the subject, comparing the amount of ET-1 and cTnI in the sample to threshold concentrations representing concentrations of ET-1 and cTnI in the blood, serum or plasma of a population of healthy patients, and determining that the subject has CHF when the concentrations of ET-1 and cTnI in the sample are above the threshold concentrations.

In another aspect, the disclosure relates to a method for detecting CHF in a subject, comprising detecting the concentration of ET-1 and cTnI in a blood, serum, or plasma sample from the subject, creating a CHF score based upon the concentrations of ET-1 and cTnI; and determining CHF, hospital admission for CHF, or relative time to hospital admission for CHF, in the subject when the score is greater than a predetermined CHF score. In some embodiments, the score comprises an odds ratio or a hazard ration. In another embodiment, the odds ratio is determined by predicting the presence or severity of CHF by the coded risk categories in a logistic regression model. The coded risk categories may be, for example, age and gender. In another example, the CHF score is determined using an area under receiver operator characteristic (AuROC) analysis. In other embodiments other biomarkers can be used in place of or in addition to cTnI. For example, a CHF score can be generated with ET-1 and any one or more of cTnI, cTnT, BNP, proBNP, NT-proBNP, IL-6, VEGF and TNF-α.

In one embodiment, to evaluate the diagnostic performance of a particular set of biomarkers, a ROC curve is generated for each biomarker. An “ROC curve” as used herein refers to a plot of the true positive rate (sensitivity) against the false positive rate (specificity) for a binary classifier system as its discrimination threshold is varied. A ROC curve can be represented equivalently by plotting the fraction of true positives out of the positives (TPR=true positive rate) versus the fraction of false positives out of the negatives (FPR=false positive rate). Each point on the ROC curve represents a sensitivity/specificity pair corresponding to a particular decision threshold.

AUC or AuROC represents the area under the Receiver Operator Characteristic curve. The AUC is an overall indication of the diagnostic accuracy of 1) a biomarker or a panel of biomarkers and 2) a ROC curve. AUC is determined by the “trapezoidal rule.” For a given curve, the data points are connected by straight line segments, perpendiculars are erected from the abscissa to each data point, and the sum of the areas of the triangles and trapezoids so constructed is computed. In certain embodiments of the methods provided herein, a biomarker protein has an AUC in the range of about 0.75 to 1.0. In certain of these embodiments, the AUC is in the range of about 0.8 to 0.9, 0.9 to 0.95, or 0.95 to 1.0.

In one embodiment, the measurement of differences in the biomarker concentrations, either up- or down-regulated, singly or in combination, in CHF patients at a particular time point, for example, 1, 2, 3, 6, 9, 12, 15, 20, 24, 30, 36, 42, and 48 months or longer, versus baseline, provides opportunities for better (e.g., simpler, earlier, faster) disease diagnosis, disease staging, risk classification, disease progression, disease severity, identification of therapy responders/non-responders, and/or the determination of the likelihood of hospitalization for CHF.

These and other methods of analysis may be used in accordance with the disclosure to provide a method for determining the likelihood of hospital admission for heart failure. The method includes determining the concentration of ET-1 and the concentration of one or more of biomarkers selected from cardiac troponin, VEGF, BNP, proBNP, NT-proBNP, and IL-6 and TNF-α in a blood, serum or plasma sample from the patient. The concentration of ET-1 in the sample is compared to the concentration of ET-1 in blood, serum or plasma samples from a population of normal healthy control subject. The concentration of the one or more biomarkers in the sample is compared to the concentration of the at least one or more biomarkers in samples from a population of normal healthy control subjects. The likelihood of hospital admission is determined when at least one of the concentration of ET-1 and the concentration of the one or more biomarkers in the patient sample are elevated above the normal healthy concentrations.

The comparing can be conducted by creating or identifying a CHF score. For example the CHF score includes an odds ratio related to the concentration of ET-1 and one or more biomarkers in a population of patients suffering from heart failure. In one embodiment, the odds ratio is determined by predicting the presence of CHF by the coded risk categories in a logistic regression model. The coded risk categories may include, for example, at least one of age, gender and hospital admission.

In another embodiment, the disclosure provides a method of determining the likelihood of a hospital admission for heart failure in a patient diagnosed with heart failure. The method includes determining the concentration of ET-1 and the concentration of one or more biomarkers selected from cardiac troponin, VEGF, BNP, proBNP, NT-proBNP, TNF-α and IL-6 in a blood, serum or plasma sample from the patient. The method also includes determining an odds ratio for ET-1 and the one or more biomarkers for the likelihood of hospital admission for the patient. The odds ratio includes a cut-off value representing the concentration of ET-1 and the one or more biomarkers in a population of heart failure patients that have been admitted to the hospital. The concentration of ET-1 and the one or more biomarkers in the sample are compared to the cut-off value, thereby determining the likelihood of hospital admission for the patient. The odds ratio may be adjusted for age and sex. The cut-off value may be determined with the empirical distribution function, or by using, for example, the mean, the 70^{th} percentile, or another appropriate value as generally understood to one of skill in the art.

In yet another embodiment, the concentration of ET-1 and the concentration of one or more of biomarkers selected from of cardiac troponin, VEGF, BNP, proBNP, NT-proBNP, TNF-α and IL-6 in a blood, serum or plasma sample from a CHF patient can be used to determine the likelihood of the patient being hospitalized for CHF sooner relative to other CHF patients. In this embodiment, a hazard ratio for hospitalization can be used to predict the likelihood of hospitalisation more quickly based on biomarker levels compared to a cut-point based upon the levels of ET-1 and the biomarkers in a healthy control population or a population of patients that have been previous diagnosed or hospitalized for heart failure.

Treatment of CHF

After congestive heart failure is diagnosed, it is usually recommended that treatment start immediately. Important, yet often neglected, aspects of treatment involves lifestyle modifications, such as reduction of sodium intake, regulation of fluid consumption, maintaining an appropriate body weight, and exercise.

In addition, CHF patients can be treated with several medications. For example, anticoagulants (e.g., Dalteparin (Fragmin), Danaparoid (Orgaran) Enoxaparin (Lovenox) Heparin (various) Tinzaparin (Innohep) and Warfarin (Coumadin)); antiplatelet agents (e.g., Aspirin, Ticlopidine, Clopidogrel (Plavix®), and Dipyridamole), Angiotensin-Converting Enzyme (ACE) Inhibitors (e.g., Benazepril (Lotensin), Captopril (Capoten), Enalapril (Vasotec), Fosinopril (Monopril), Lisinopril, (Prinivil, Zestril), Moexipril (Univasc), Perindopril (Aceon), Quinapril (Accupril), Ramipril (Altace), Trandolapril (Mavik); Angiotensin II Receptor Blockers (or inhibitors) (e.g., Candesartan (Atacand), Eprosartan (Teveten), Irbesartan (Avapro), Losartan (Cozaar), Telmisartan (Micardis), and Valsartan (Diovan); Beta Blockers (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren); Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine, (Nimotop), Nisoldipine (Sular), Verapamil (Calan, Isoptin, Verelan); Diuretics (e.g., Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and Spironolactone Aldactone); Vasodialators (e.g., Isosorbide dinitrate (Isordil), Nesiritide (Natrecor), Hydralazine (Apresoline), Nitrates, Minoxidil); Digitalis Preparations (Digoxin and Digitoxin, e.g., Lanoxin); and Statins (e.g., statins, resins, nicotinic acid (niacin), gemfibrozil, clofibrate etc.).

Accordingly, in various aspects, the disclosure is directed to a method for treating CHF, preventing CHF, preventing or slowing the progression of CHF, and/or prolonging or preventing hospital admission or death following a diagnosis of CHF. The method includes determining the risk or severity of CHF, or the likelihood of hospital admission, according to the methods of this disclosure, and treating the patient that has a risk of CHF, or has CHF, with the above treatment options. Such treatment can prevent CHF, prevent or slow the progression of CHF, and avoid or prolong hospital admission for CHF.

Instruments and Systems Suitable for Highly Sensitive Analysis of Biomarkers

As noted above, the diagnostic/prognostic methods described herein generally involve the determination of the amount of one or more biomarkers related to CHF from one or a set of samples from a subject. Determination of concentrations of biomarker related to CHF in the practice of the methods can be performed using any suitable apparatus or system that allow for the detection levels described herein. See, e.g., U.S. Pat. Nos. 7,838,250, 7,572,640, and 7,914,734, which are incorporated by reference herein in their entireties. These patents describe instruments, reagents and methods for measuring analytes at levels to carry out the methods of the disclosure and thus identify those patients with biomarker levels related to CHF or CHF progression.

In one example, an automatic sampling system may be included in the analyzer system for introducing the sample into the analyzer system. In another embodiment of the analyzer system, a sample preparation system may be included in the analyzer system for preparing a sample. In a further embodiment, the analyzer system may contain a sample recovery system for recovering at least a portion of the sample after analysis is complete.

In one aspect, the analyzer system consists of an electromagnetic radiation source for exciting a single particle labelled with a fluorescent label. In one embodiment, the electromagnetic radiation source of the analyzer system is a laser. In a further embodiment, the electromagnetic radiation source is a continuous wave laser.

In an exemplary embodiment, the electromagnetic radiation source excites a fluorescent moiety attached to a label as the label passes through the interrogation space of the capillary flow cell. In some embodiments, the fluorescent label moiety includes one or more fluorescent dye molecules. In some embodiments, the fluorescent label moiety is a quantum dot.

When the interrogation space is a capillary flow cell, a label is exposed to electromagnetic radiation when the label passes through an interrogation space. The interrogation space is typically fluidly connected to a sampling system. In some embodiments the label passes through the interrogation space of the capillary flow cell due to a motive force to advance the label through the analyzer system. The interrogation space is positioned such that it receives electromagnetic radiation emitted from the radiation source. In some embodiments, the sampling system is an automated sampling system capable of sampling a plurality of samples without intervention from a human operator.

The label passes through the interrogation space and emits a detectable amount of energy when excited by the electromagnetic radiation source. In one embodiment, an electromagnetic radiation detector is operably connected to the interrogation space. The electromagnetic radiation detector is capable of detecting the energy emitted by the label, e.g., by the fluorescent moiety of the label.

In a further embodiment of the analyzer system, the system further includes a sample preparation mechanism where a sample may be partially or completely prepared for analysis by the analyzer system. In some embodiments of the analyzer system, the sample is discarded after it is analyzed by the system. In other embodiments, the analyzer system further includes a sample recovery mechanism whereby at least a portion, or alternatively all or substantially all, of the sample may be recovered after analysis. In such an embodiment, the sample can be returned to the origin of the sample. In some embodiments, the sample can be returned to microtiter wells on a sample microtiter plate. The analyzer system typically further consists of a data acquisition system for collecting and reporting the detected signal.

Sample Preparation

In certain embodiments, the patient sample must be prepared for analysis according to the methods of the disclosure.

In general, any method of sample preparation may be used that produces a label corresponding to a bimarker to be measured, where the label is detectable in the instruments described herein. As is known in the art, sample preparation in which a label is added to one or more particles may be performed in a homogeneous or heterogeneous format. In some embodiments, the sample preparation is formed in a homogenous format. In analyzer system employing a homogenous format, unbound label is not removed from the sample. See, e.g., U.S. Pat. Nos. 7,838,250, 7,572,640, and 7,914,734. In some embodiments, the particle or particles of interest are labelled by addition of labelled antibody or antibodies that bind to the particle or particles of interest.

Antibodies and Binding Partners

In one aspect of the disclosure, the biomarker to be measured, such as ET-1, cTnI, cTnT, BNP, proBNP, NT-proBNP, IL-6, VEGF and TNF-α, can be joined with a binding partner. Any suitable binding partner with the requisite specificity for the form of molecule to be detected can be used. If the molecule a marker has several different forms, various specificities of binding partners are possible. Suitable binding partners are known in the art and include antibodies, aptamers, lectins, and receptors.

In one aspect of the disclosure, the amount of ET-1, cTnI, cTnT, BNP, proBNP, NT-proBNP, IL-6, VEGF and TNF-α is determined by contacting the biological sample with an antibody specific for ET-1, cTnI, cTnT, BNP, proBNP, NT-proBNP, IL-6, VEGF and TNF-α and determining the amount of specific binding between the antibody and ET-1, cTnI, cTnT, BNP, proBNP, NT-proBNP, IL-6, VEGF and TNF-α in the sample.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various forms of the molecule, if present, or for total (e.g., all, or substantially all, of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments that mimic antibodies can be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn. USA; HyTest Ltd., Turku Finland; Abcam Inc., Cambridge, Mass., USA, Life Diagnostics, Inc., West Chester, Pa., USA; Fitzgerald Industries International, Inc., Concord, Mass. USA; BiosPacific, Emeryville, Calif. USA).

In some embodiments, the antibody is a polyclonal antibody. In other embodiments, the antibody is a monoclonal antibody.

Capture binding partners and detection binding partner pairs, e.g., capture and detection antibody pairs, can be used in embodiments of the disclosure. Thus, in some embodiments, a heterogeneous assay protocol is used in which, typically, two binding partners, e.g., two antibodies, are used. One binding partner is a capture partner, usually immobilized on a solid support, and the other binding partner is a detection binding partner, typically with a detectable label attached. Such antibody pairs are available from several commercial sources, Antibody pairs can also be designed and prepared by methods well-known in the art. Compositions of the disclosure include antibody pairs wherein one member of the antibody pair is a label as described herein, and the other member is a capture antibody.

Antibodies to ET-1, cTnI, cTnT, BNP, proBNP, NT-proBNP, IL-6, VEGF and TNF-α are well characterized in the field of the disclosure. Antibodies to ET-1, cTnI, cTnT, BNP, proBNP, NT-proBNP, IL-6, VEGF and TNF-α are available from a variety of commercial and non-commercial sources. The disclosure is not limited to any of the particular antibodies provided herein for exemplary purposes.

### EXAMPLES

### Example 1

The ERENNA® System, based upon Singulex Single Molecule Counting technology, was used for immunoassay analysis. This system has been described in Todd, et al., Clin Chem. 53(11): 1990-1995 (2007); Todd et al., Clin Chem. 55(1):196-8 (2009) and U.S. Pat. No. 7,572,640, which is incorporated by reference in its entirety.

The 21 amino acid ET-1 analyte was obtained for various commercial vendors. Antibodies for the ET-1 immunoassays, MA10818, MA3440, and MA3005 were obtained from R&D Systems, Inc. (Minneapolis, Minn.) and Thermo Scientific (Middleton, Va.). The cTnI analyte was obtained from HyTest (Turku, Finland).

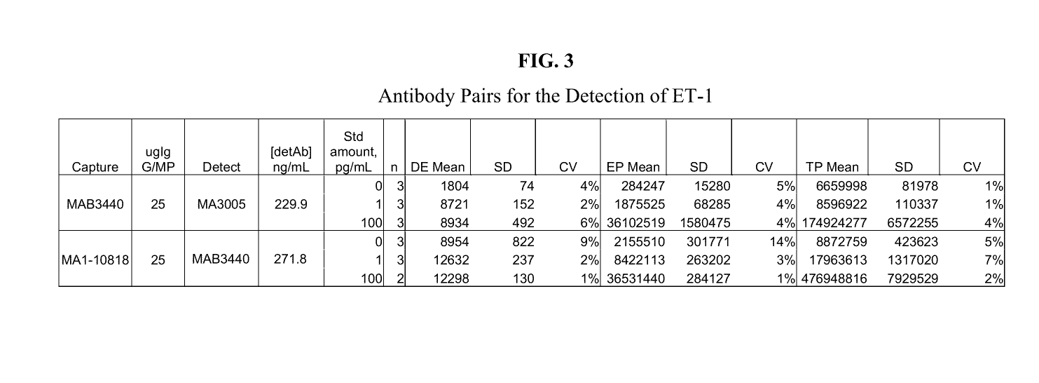

Antibody pairs for the detection of ET-1 were determined through a series of experiments in the manner outlined below with the commercially available antibodies and the ET-1 analyte. Antibody pairs (1) MAB3440 (Capture) and MA3005 (Detection), and (2) MA1-10818 (capture) and MAB3440 (detection) were chosen as providing the highest sensitivity with the lowest coefficient of variation, which is shown in FIG. 3 for these pairs.

Analysis of blood samples for ET-1 were conducted with the ERENNA® system as follows: 100 μL assay buffer with paramagnetic microparticles (MPs, 1 μm, DYNABEADS® MYONE™, Life Technologies) coated with capture antibody added to each well in a 96 well plate. 100 uL assay ET-1 standard or biological samples (e.g. human plasma) were added to each well in plate. The plate was covered and incubated for 2 hours at 25° C. with shaking. The plate was then washed to remove unbound material, and a magnetic bed (Ambion) was used to retain the MPs in well. 20 μL detection antibody (ALEXA FLUOR® labelled) were added to each well. The plate was covered and incubated at 25° C. for 1 hour with shaking. The plate was washed to remove unbound detection antibody. The magnetic bed was used to retain MPs in the well.

The MPs were transferred to a fresh 96 well plate and the bound detection antibody was released from MPs via elution with 10 μL glycine. Glycine buffer containing ALEXA FLUOR® dye labelled detection antibody was transferred to 384 well receptacle plate containing 10 uL 0.5M TRIS buffer (to neutralize glycine). The MPs were retained in the 96 well plate via magnetic separation using the magnetic bed. The 384 well plate was read in the ERENNA™ system to count individual ALEXA FLUOR® dye labelled detection antibody molecules from each well.

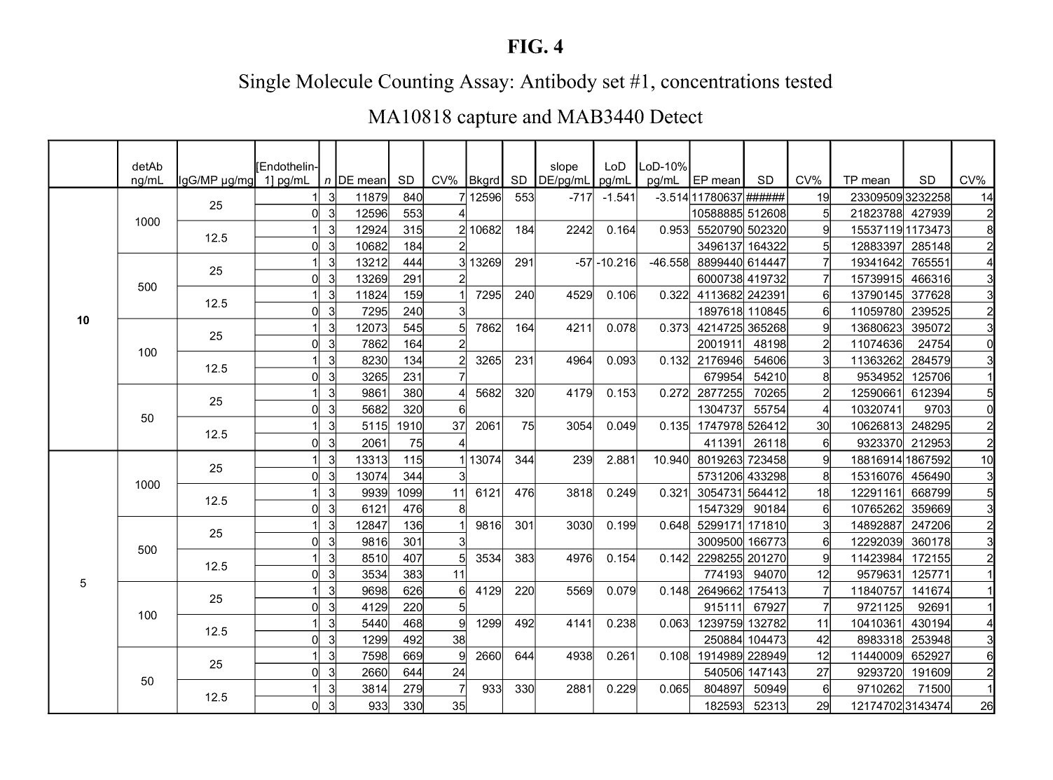

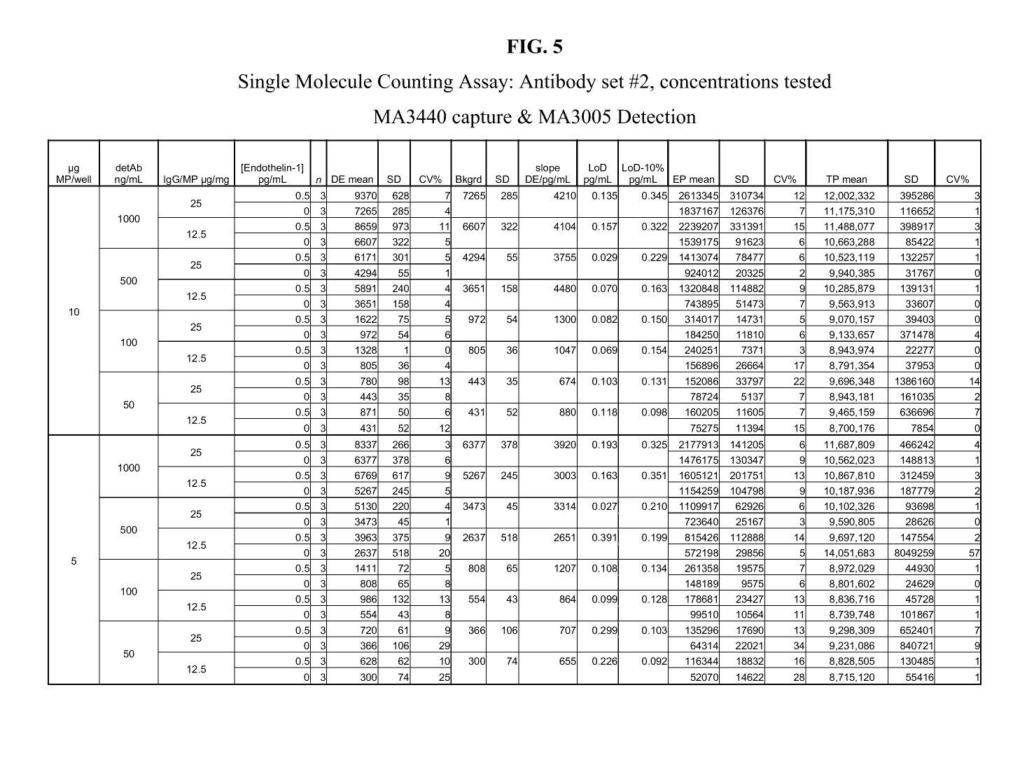

Using the ERENNA® System, an ET-1 assay was developed using antibodies MAB3440 (Capture) and MA3005 (Detection). The concentrations of antibodies on the microparticles and in the detection reagents for the ET-1 assays were optimized in experiments using the method described above and the concentration of antibodies and microparticles shown in FIGS. 4 and 5.

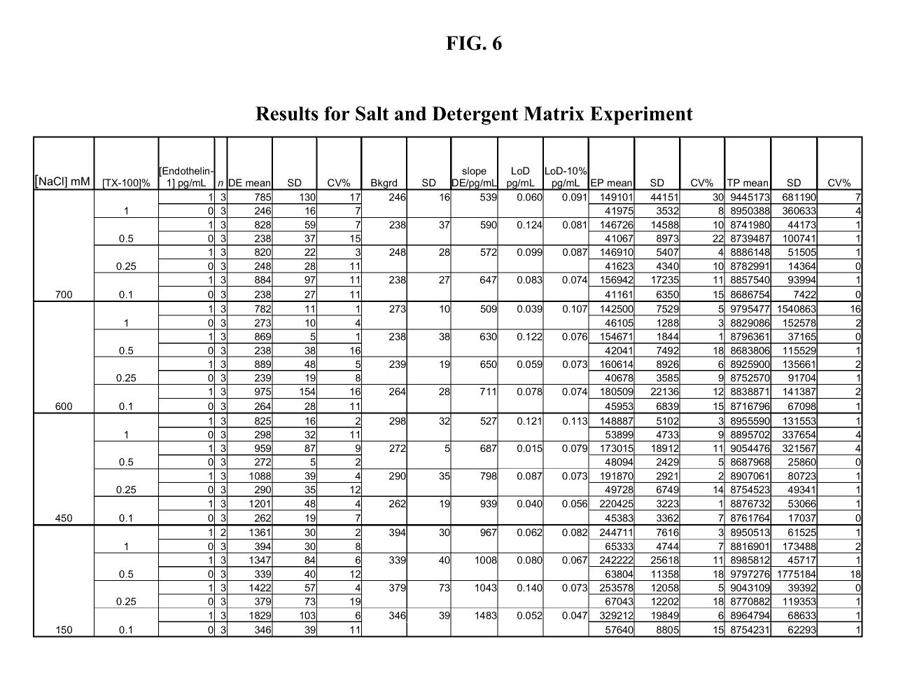

Optimal salt and detergent concentrations in assays for ET-1 were determined experimentally as shown in FIG. 6. The best conditions were determined with the general assay buffer at 150 mM NaCl/0.1% Triton and MPs at 10 ug/well.

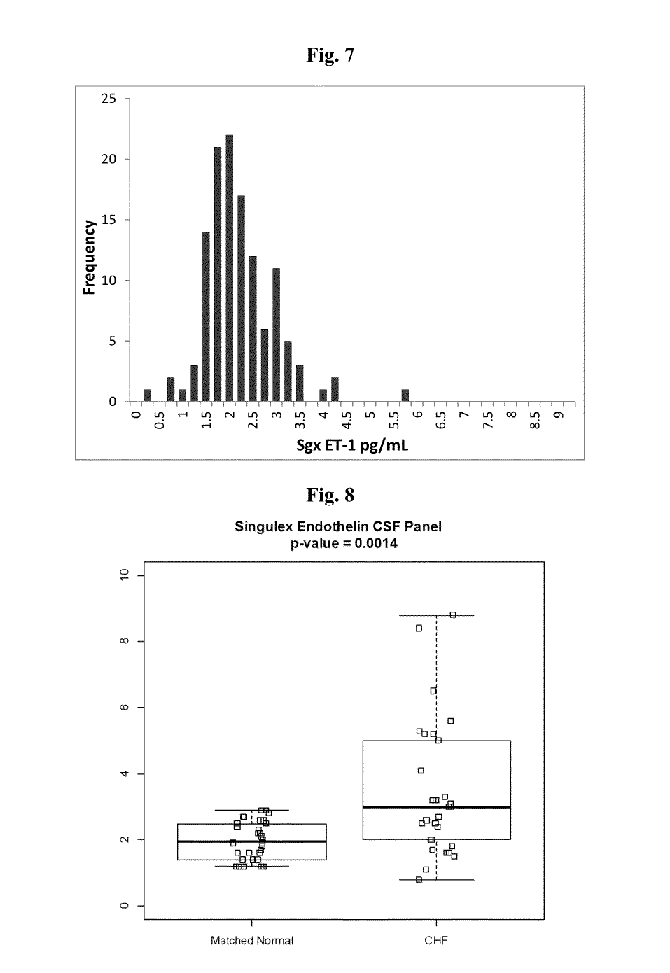

FIG. 7 shows the distribution of ET-1 blood concentrations in populations of healthy volunteers using the Sgx ET-1 assay. FIG. 8 shows the difference in the medians in ET-1 concentration in blood samples from the age-matched healthy and CHF populations shown in FIG. 2 using the Sgx ET-1 assay.

### Example 2

The detection of cTnI was accomplished with the ERRENA system (Singulex, Inc.) as generally described in U.S. Pat. No. 8,343,728, which is incorporated by reference in its entirety. Antibodies for cTnI were obtained from BiosPacific (Emeryville, Calif.).

Briefly, the sample was added to a well in a 96 well plate, along with sufficient volume of calibrator diluent (3% BSA, Tris pH 8.0, 150 mM NaCl) to create a final volume of 50 pt. 150 μL of paramagnetic microparticles (MPs, MyOne, Invitrogen Dynal AS; approximately 5-10 ug MPs/well), coated with the capture antibody and diluted in assay buffer (1% BSA, Tris-buffered saline, pH 7.4, with 0.5 mL Triton X-100/L, and heterophile/human antimouse antibody-blocking reagents (from Scantibodies Laboratories, used per the manufacturer's recommendations), were added to each well and incubated for about 1 hour. MPs were separated using a magnetic bed (Ambion). Supernatant was removed, MPs were washed once, and then 20 μL of ALEXA FLUOR® dye labelled detection antibody (50-500 mg/L diluted in assay buffer) was added and incubated for about 1 hour at 25° C. with shaking. The MPs were again magnetically separated and washed 5 times using Tris-buffered saline with 0.5 mL Triton X-100/L. After removal of residual wash buffer, 20 μL elution buffer (Glycine pH 2.5) was added. This reagent disrupted antibody-analyte interactions and resulted in the release of detection antibody from the MPs. The solution in each 96-well plate was then transferred to a 384-well filter plate (0.2 um, AcroPrep cat. no. 5070, Pall) and centrifuged at 1200 g for 3 minutes to separate detection antibody in elution buffer from MPs. The eluted and filtered material in the 384-well plate was then placed into the ERENNA® Immunoassay System. The concentration of cTnI in each sample was determined via interpolation off a standard curve run with the samples.

Using a cutpoint of 6 pg/mL for cTnI and a cutpoint of either 3 pg/mL or 2 pg/mL for ET-1, and determining the number of HF or control samples that provided biomarker (either separate or combined) values above this cutpoint, the sensitivity and specificity of diagnosing HF was determined. Using the cutpoint of 6 pg/mL for cTnI and 3 pg/mL for ET-1, the sensitivity was 60% and the specificity was 90%. Using the cutpoint of 6 pg/mL for cTnI and 2 pg/mL for ET, the sensitivity was 87% and the specificity was 83%. These findings demonstrate that cTnI and ET can be used to diagnose HF.

### Example 3

Measurements of TNF-α and VEGF were determined using the ERENNA® System in an assay configuration similar to the ET-1 and cTnI assays. The VEGF and TNF-α assays have been described previously. See Todd, et al. Clin Chem (supra) and U.S. Pat. No. 8,450,069. Antibody pairs were as follows:

VEGF

Capture: ab36424 (Abcam)

Detection: ab17696 (Abcam)

TNF-alpha

Capture: MAB610 (R&D Systems)

Detection: AF-210-NA(R&D Systems)

121 heart failure patients having NYHA classifications of 2 or 3 (Ejection Fraction<40%, Age=59+/−11 years) were followed from 1.4 to 3.4 years following hospital discharge. Baseline and 3 months post discharge blood draws were tested for ET-1 with a kit from R&D Systems, Inc. and cTnI as described above. Patients were also classified into one of four groups (1) death, (2) rehospitalisation for heart failure, (3) rehospitalisation for any cause, and (4) no rehospitalisation. Odds ratios of hospitalization for HF were determined using the baseline biomarker values and statistical software SAS 9.3 (SAS Institute, Inc).

In addition, odds ratios were determined with the individual biomarkers VEGF and TNF-alpha as shown in Table 1, which can then be introduced into a combined ET and cTnI odds ratio. The odds ratios were determined after adjusting for age, sex and the other biomarkers in Table 1. Since they were adjusted for the other biomarkers and retained statistical significance, it is reasonable to expect that there will be synergy among them.

TABLE 1
Biomarkers dichotomized at “At Risk” Cut Point

| Marker | At Risk |
|---|---|
| Singulex cTnI | 8.24 (2.56-26.53) |
| R&D Systems ET-1 | 6.97 (2.15-22.55) |
| Singulex VEGF | 4.09 (1.39-11.98) |
| Singulex TNFα | 2.95 (1.04-8.32) |

The odds ratio for for predicting rehospitalization for HF using a combination of ET and cTnI was 17.3 (95% CI:4.3-69.4). This means that a HF patient with elevated cTnI and ET has 17.3 times higher odds of rehospitalization than a patient with only one high biomarker or with neither biomarker elevated above the cutpoint.

### Example 4

134 heart failure patients were followed for up to three years after entering a study for heart failure hospitalization (or rehospitialization) following heart failure diagnosis (144 median days between diagnosis and study entry). Baseline (study entry) and interval plasma samples were collected and frozen for later analysis.

Single molecule counting assays on the ERENNA® system used the following antibody pairs:

Sgx1 ET1 Assay:

Capture: R&D Systems Monoclonal MAB3440

Detection: Pierce Monoclonal MA3-005

Sgx2 ET1 Assay:

Capture: R&D Systems Monoclonal MAB3440

Detection: Millipore Monoclonal CBL-85

Samples were also analyzed for other biomarkers. Analysis for BNP and NT-proBNP were conducted with commercially available kits. C-terminal-proET-1 (CT-proET-1) analysis was conducted with a kit from (B.R.A.M.H.S.). IL-6, TNF-α, and VEGF were tested in single molecule counting assays using the ERENNA® system (Singulex, Inc.) as described above and in U.S. Pat. No. 8,450,069. The antibody pair for IL-6 were as follows: capture MAB206 (R&D Systems), detection AF-206-NA (R&D Systems).

Table 2 shows the biomarker plasma concentrations for patients that were hospitalized and patients that were not hospitalized during the 1500 day study (all values in pg/mL except for CT-proET1, which is pmol/mL).

TABLE 2

| | Hospitalized for | Hospitalized for | |
|---|---|---|---|
| | HF (NO) | HF (YES) | |
| Biomarker | (median) | (median) | p-value |
| Age | 58 | 57.5 | 0.9165 |
| BNP | 222 | 537 | <.0001 |
| NTproBNP | 1010 | 3058 | <.0001 |
| IL6 | 3.1 | 5.1 | 0.0213 |
| TNFa | 4.4 | 6.0 | 0.0111 |
| cTnI | 7.7 | 14.6 | 0.0026 |
| VEGF | 35.3 | 47.9 | 0.0224 |
| CT-proET1 | 73 | 111 | <.0001 |
| Sgx1_ET1 | 4.2 | 5.3 | 0.0007 |
| Sgx2_ET1 | 4.3 | 5.2 | 0.0035 |

Table 3 shows the cut-off value for an odds ratio analysis to determine the likelihood of hospitalization. The cut-off values represent well-known cut points identified in the literature as low or high cut-off values for the diagnosis of heart failure. When literature references are not available or are not consistent, the median was used as a low cut-off, and the 75th percentile was used as the high cut-off value. EDF represents the empirical distribution function used to determine an optimal cut-off value.

TABLE 3

| | Median or | 75th % tile or | |
|---|---|---|---|
| Biomarker | literature | literature | EDF |
| IL-6 | 3.3 | 6.9 | 14 |
| TNFa | 4.7 | 6.9 | 5.3 |
| VEGF | 39.5 | 62.0 | 35.4 |
| CT-proET-1 | 79.0 | 113.4 | 80 |
| Sgx1_ET1 | 4.4 | 5.8 | 6.5 |
| Sgx2_ET1 | 4.5 | 5.8 | 6.5 |
| cTnI | 9.5 | 18.4 | 6.8 |
| BNP | 100 | 400 | 1200 |
| NTproBNP | 125 | 450 | 1000 |
| CRP | 1 | 3 | 16 |

Table 4 shows the unadjusted (not adjusted for age and/or sex) odds ratios for individual biomarkers for predicting hospitalization for heart failure. Cut-off values are identified in Table 3.

TABLE 4
Odds rations (95% confidence intervals)

| Marker | Median | 75^{th} % tile | EDF |
|---|---|---|---|
| CT-proET1 | 9.5 (3.4, 26.5) | 4.0 (1.7, 9.4) | 9.9 (3.5, 27.7) |
| Sgx1_ET1 | 4.5 (1.9, 10.9) | 3.0 (1.3, 6.8) | 5.0 (2.0, 12.7) |
| Sgx2_ET1 | 3.5 (1.5, 8.3) | 3.0 (1.3, 6.8) | 4.5 (1.8, 11.4) |
| cTnI | 4.5 (1.9, 10.9) | 1.6 (0.7, 3.7) | 6.9 (2.3, 21.1) |
| BNP | 4.0 (1.1, 14.2) | 5.1 (2.2, 11.8) | 4.2 (1.5, 15.1) |
| NTproBNP | 3.2 (0.4, 26.7) | 4.0 (1.1, 14.2) | 4.5 (1.7, 11.8) |
| TNFa | 2.3 (1.0, 5.4) | 2.6 (1.1, 5.8) | 3.4 (1.5, 7.7) |
| VEGF | 2.6 (1.1, 5.8) | 2.0 (0.9, 4.8) | 4.7 (1.8, 12.2) |
| IL-6 | 2.6 (1.1, 5.7) | 3.0 (1.3, 7.0) | 4.0 (1.3, 13.1) |

Table 5 shows the odds ratio for each of CT-proET-1, Sgx ET-1. and cTnI to independently predict hospitalization when adjusted for each other, age, and sex. In the analyses, the EDF was used as the cut-off value.

TABLE 5

| | | |
|---|---|---|
| Biomarker | OR | 95% Confidence Interval |

| | | | |
|---|---|---|---|
| CT-proET1 | 10.1 | 3.3 | 31.0 |
| Sgx1 ET1 | 3.7 | 1.3 | 10.7 |

| | | |
|---|---|---|
| Biomarker | OR | 95% CI |

| | | | |
|---|---|---|---|
| CT-proET1 | 8.3 | 2.7 | 25.4 |
| cTnI | 4.4 | 1.3 | 15.2 |
| Sqx1-ET1 | 3.8 | 1.4 | 10.3 |
| cTnI | 6.4 | 1.9 | 21.0 |

Tables 6 and 7 show the ability of individual biomarkers to predict HF hospitalization in a multimarker analysis. Elevated levels of ET-1, cTnI, and VEGF independently predicted HF hospitalization and all were statistically significant. The EDF cut-off was used in the analyses. Similar results were obtained by replacing BNP with NT-proBNP or using different cut-off points.

TABLE 6

| Biomarker | OR | 95% CI | |
|---|---|---|---|
| NTproBNP* | 1.3 | 0.4 | 4.7 |
| IL-6 | 1.5 | 0.4 | 6.7 |
| TNF-a | 1.4 | 0.5 | 4.2 |
| cTnI | 4.2 | 1.1 | 16.0 |
| VEGF | 3.9 | 1.3 | 11.4 |
| CT-proET1 | 5.3 | 1.5 | 18.7 |

TABLE 7

| Biomarker | OR | 95% CI | |
|---|---|---|---|
| NTproBNP* | 1.7 | 0.5 | 5.6 |
| IL-6 | 1.4 | 0.3 | 6.2 |
| TNF-a | 2.2 | 0.8 | 6.0 |
| cTnI | 4.2 | 1.1 | 15.3 |
| VEGF | 2.4 | 1.0 | 6.2 |
| Sgx ET1 | 2.9 | 1.0 | 7.9 |

### Example 5

Analyses were conducted with various combinations of biomarkers to determine likelihood of hospital admission for the patient population discussed in Example 4. In this example, “survival” refers to the likelihood of a patient avoiding hospitalization or death following a diagnosis and/or prior hospitalization for heart failure.

Table 8 shows the hazard ratios for hospitalization, which predicts likelihood of hospitalization more quickly based on biomarker levels compared the cut-point. The data is unadjusted for age, sex or other biomarkers.

TABLE 8

| Biomarker | Hazard Ratio | P-value |
|---|---|---|
| Age | 1.0 | 0.9557 |
| Gender | 1.4 | 0.3549 |
| Sgx1 ET1 | 4.1 | <.0001 |
| CT-proET1 | 7.7 | <.0001 |
| IL6 | 3.9 | 0.0015 |
| TNFA | 3.0 | 0.0020 |
| VEGF | 4.0 | 0.0021 |
| cTnI | 5.6 | 0.0012 |
| BNP | 3.7 | 0.0012 |
| NTproBNP | 3.9 | 0.0026 |

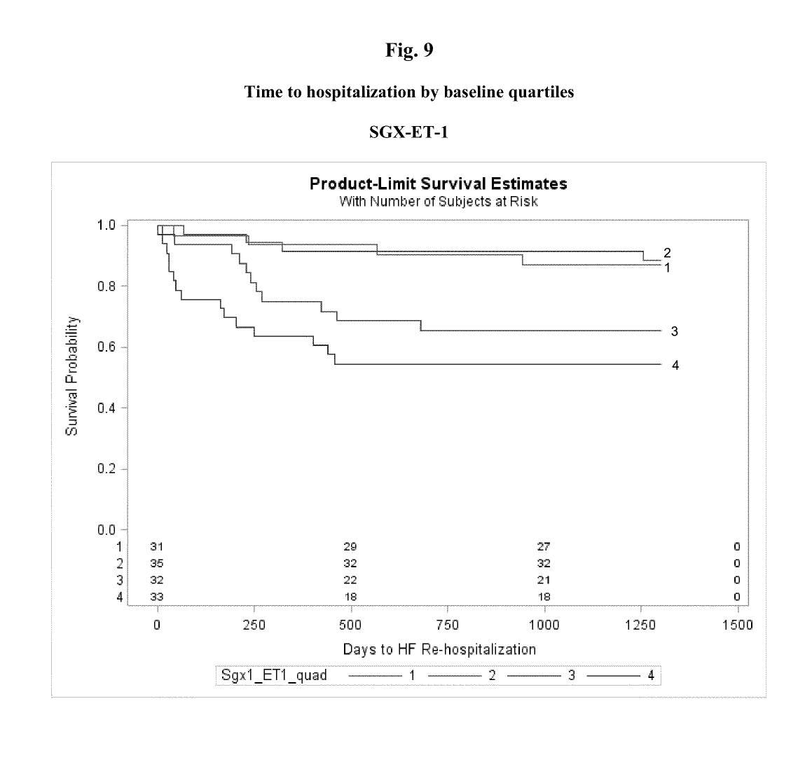

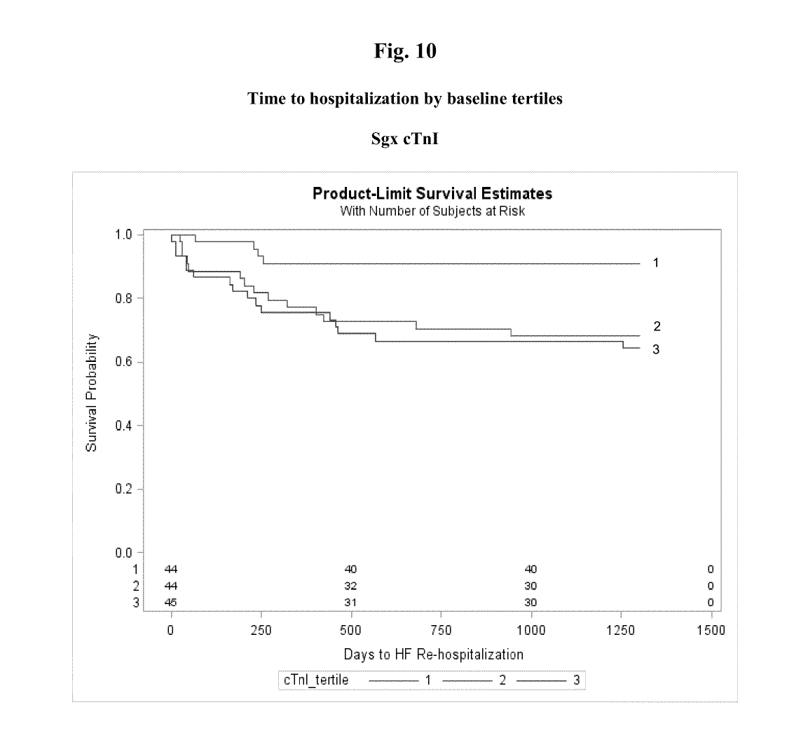

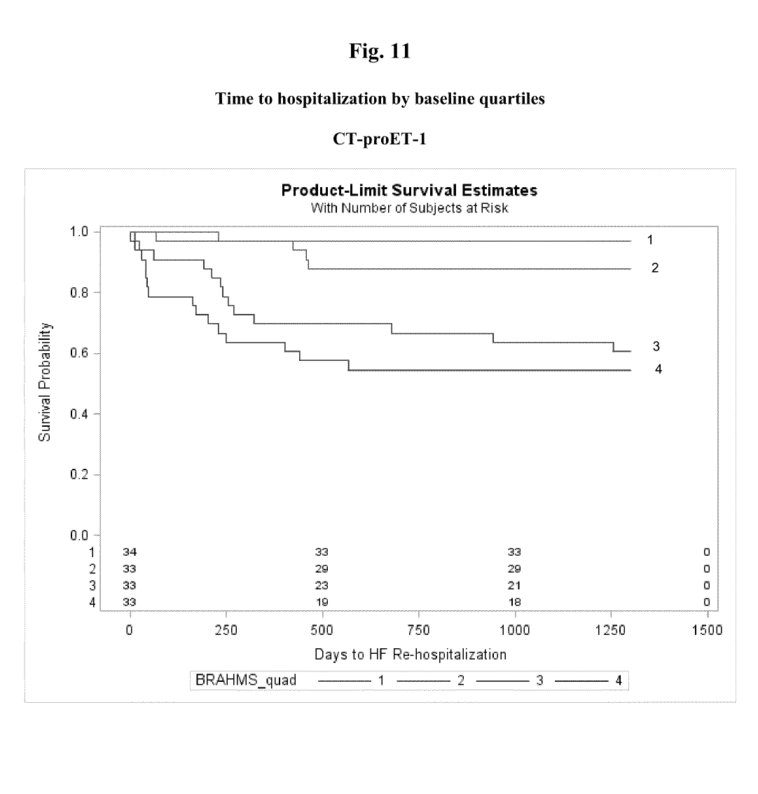

FIGS. 9, 10 and 11 demonstrate that patients in the higher tertiles or quartiles of Sgx ET1 (FIG. 9), cTnI (FIG. 10), or CT-proET1 (FIG. 11) have a higher likelihood of hospitalization sooner than patients in lower tertiles or quartiles. Patients were followed for 1300 days.

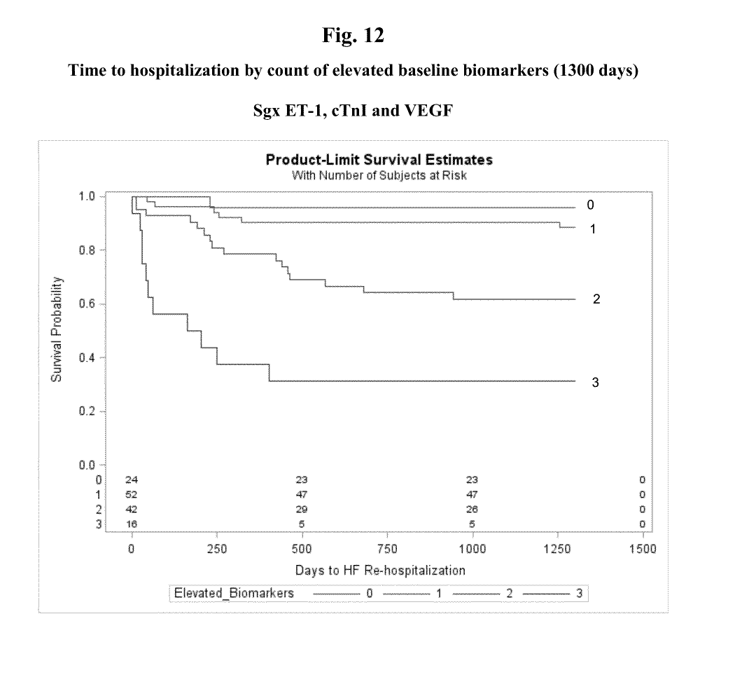

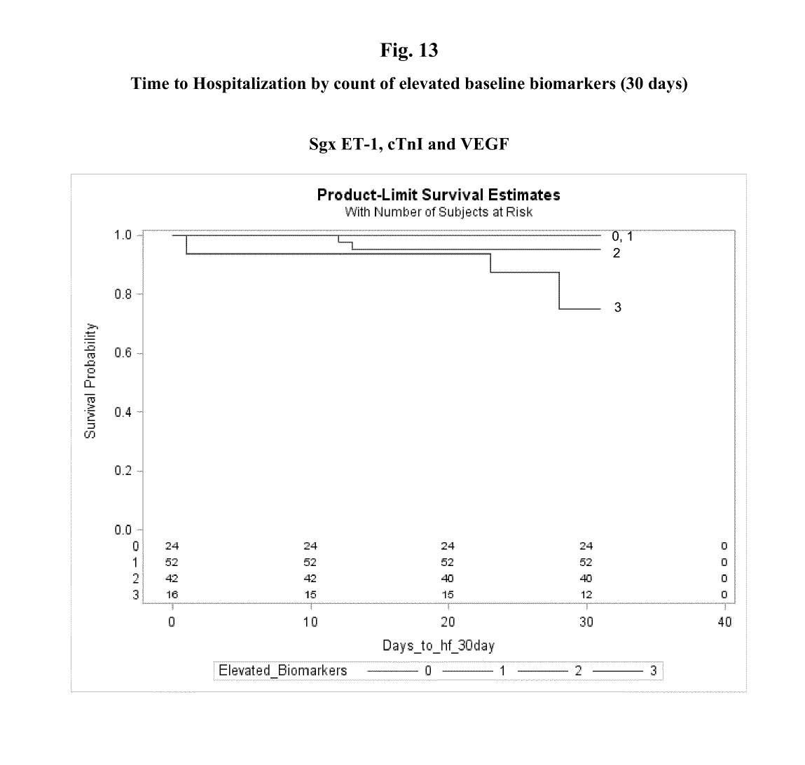

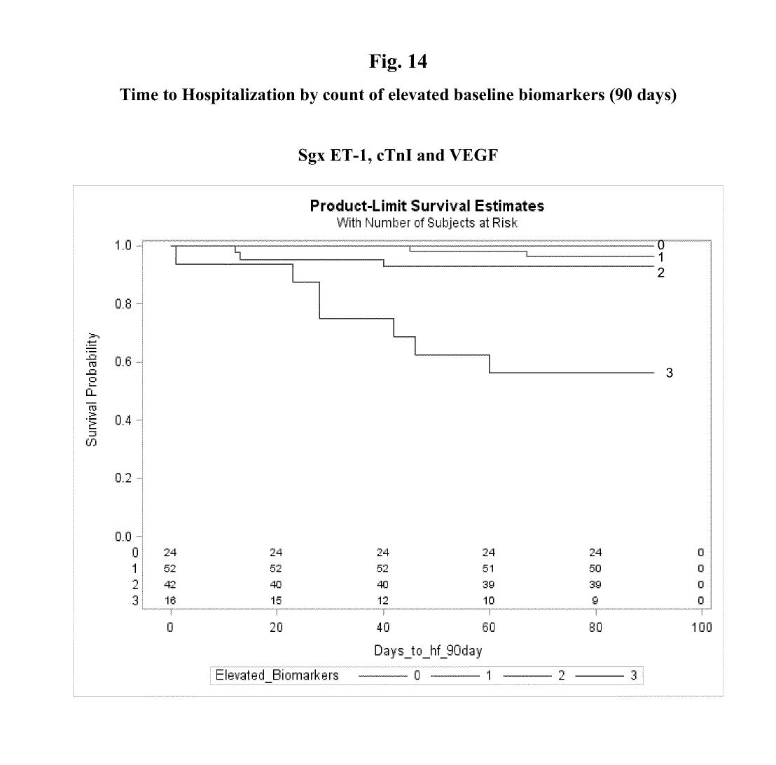

FIGS. 12, 13 and 14 demonstrate that patients with a higher number of elevated biomarkers (Sgx ET1, cTnI, or VEGF) have an increased likelihood of hospitalization quicker than patients with a lower number of elevated biomarkers. Patients were followed for 1300 days (FIG. 12), 30 days (FIG. 13) and 90 days (FIG. 14).

Although various specific embodiments of the present disclosure have been described herein, it is to be understood that the disclosure is not limited to those precise embodiments and that various changes or modifications can be affected therein by one skilled in the art without departing from the scope and spirit of the disclosure.

The examples given above are merely illustrative and are not meant to be an exhaustive list of all possible embodiments, applications or modifications of the disclosure. Thus, various modifications and variations of the described methods and systems of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure which are obvious to those skilled in molecular biology, immunology, chemistry, biochemistry or in the relevant fields are intended to be within the scope of the appended claims.

It is understood that the disclosure is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the disclosure.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the disclosure. The disclosures of all references and publications cited herein are expressly incorporated by reference in their entireties to the same extent as if each were incorporated by reference individually.

1. A method for determining the risk or severity of cardiac disease, the method comprising:
determining the concentration of endothelin 1 (ET-1) and at least one of cardiac troponin, Vascular Endothelial Growth Factor (VEGF), Tumor Necrosis Factor alpha TNF-α, Brain Natriuretic Peptide (BNP), proBNP, N-terminal proBNP (NT-proBNP), Interleukin-6 (IL-6), in a blood, serum or plasma sample from a patient, and using the concentrations to predict the risk or severity of cardiac disease.

2. The method of claim 1, wherein the cardiac troponin is cTnI.

3. The method of claim 1, wherein the cardiac disease is cardiac heart failure (CHF).

4. The method of claim 1, wherein the patient has been hospitalized for heart failure.

5. The method of claim 1, wherein the concentrations of (ET-1) and at least one of cardiac troponin, VEGF, IL-6, BNP, proBNP, NT-proBNP, and TNF-α in a blood sample from a patient are compared to the concentrations in a population of normal healthy control subjects.

6. The method of claim 1, wherein the concentrations of ET-1 and at least one of cardiac troponin, VEGF, IL-6, BNP, proBNP, NT-proBNP, and TNF-α in a blood sample from a patient are compared to the concentrations in a population of patients that have been admitted to the hospital for cardiac heart failure.

7. The method of claim 3, wherein the risk or severity of cardiac disease is the likelihood of admission to a hospital following the diagnosis of cardiac heart failure.

8. A method for determining the likelihood of hospital admission for heart failure; the method comprising;
determining the concentration of ET-1 and the concentration of one or more of biomarkers selected from the group consisting of cardiac troponin, VEGF, BNP, proBNP, NT-proBNP, TNF-α and IL-6 in a blood, serum or plasma sample from the patient;
comparing the concentration of ET-1 in the sample to the concentration ET-1 in blood, serum or plasma samples from a population of normal healthy control subjects;
comparing the concentration of the one or more biomarkers in the sample to the concentration of the at least one or more biomarkers in samples from a population of normal healthy control subjects; and
determining the likelihood of hospital admission when at least one of the concentration of ET-1 and the concentration of cTnI in the patient sample is elevated above the normal healthy concentrations.

9. The method of claim 8, wherein the comparing comprises identification of a CHF score.

10. The method of claim 9, wherein the score comprises an odds ratio related to hospital admission following a diagnosis of heart failure or previous hospital admission for heart failure.

11. The method of claim 8, wherein the score comprises an odds ratio.

12. The method of claim 9, wherein the odds ratio is determined by predicting the presence of CHF by the coded risk categories in a logistic regression model.

13. The method of claim 9, wherein the coded risk categories include at least one of age, gender and hospital admission.

14. A method of determining the likelihood of a hospital admission for heart failure in a patient diagnosed with heart failure, the method comprising:
determining the concentration of ET-1 and the concentration of one or more of biomarkers selected from the group consisting of cTnI, cTnT, VEGF, BNP, proBNP, NT-proBNP, TNF-α and IL-6 in a blood, serum or plasma sample from the patient;
determining an odds ratio for ET-1 and the one or more biomarkers for the likelihood of hospital admission for the patient, wherein the odds ratio comprises a cut-off value representing the concentration of ET-1 and the one or more biomarkers in a population of heart failure patients that have been admitted to the hospital, and
comparing the concentration of ET-1 and the one or more biomarkers in the sample to the cut-off value, thereby determining the likelihood of hospital admission for the patient.

15. The method of claim 14, wherein the more or more biomarkers comprises cTnI.

16. The method of claim 14, wherein the odds ratio is adjusted for age and sex.

17. A method of treating CHF comprising determining that a patient is suffering from CHF using a method according to any of claims 1-7, and administering a treatment to the patient comprising regulating the subjects diet, fluid intake, sodium intake and exercise, or treating the patient with one or more of the following compounds: anticoagulants (e.g., Dalteparin (Fragmin), Danaparoid (Orgaran) Enoxaparin (Lovenox) Heparin (various) Tinzaparin (Innohep) and Warfarin (Coumadin)); antiplatelet agents (e.g., Aspirin, Ticlopidine, Clopidogrel (Plavix®), and Dipyridamole), Angiotensin-Converting Enzyme (ACE) Inhibitors (e.g., Benazepril (Lotensin), Captopril (Capoten), Enalapril (Vasotec), Fosinopril (Monopril), Lisinopril, (Prinivil, Zestril), Moexipril (Univasc), Perindopril (Aceon), Quinapril (Accupril), Ramipril (Altace), Trandolapril (Mavik); Angiotensin II Receptor Blockers (or inhibitors) (e.g., Candesartan (Atacand), Eprosartan (Teveten), Irbesartan (Avapro), Losartan (Cozaar), Telmisartan (Micardis), and Valsartan (Diovan); Beta Blockers (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren); Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine, (Nimotop), Nisoldipine (Sular), Verapamil (Calan, Isoptin, Verelan); Diuretics (e.g., Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and Spironolactone Aldactone); Vasodialators (e.g., Isosorbide dinitrate (Isordil), Nesiritide (Natrecor), Hydralazine (Apresoline), Nitrates, Minoxidil); Digitalis Preparations (Digoxin and Digitoxin, e.g., Lanoxin); and Statins (e.g., statins, resins, nicotinic acid (niacin), gemfibrozil, clofibrate).

18. A method for preventing a hospital admission for CHF, the method comprising determining the likelihood of a hospital admission for CHF according the method of any one of claims 8-14, and administering a treatment to the patient comprising regulating the subjects diet, fluid intake, sodium intake, and exercise, or treating the patient with one or more of the following compounds: anticoagulants (e.g., Dalteparin (Fragmin), Danaparoid (Orgaran) Enoxaparin (Lovenox) Heparin (various) Tinzaparin (Innohep) and Warfarin (Coumadin)); antiplatelet agents (e.g., Aspirin, Ticlopidine, Clopidogrel (Plavix®), and Dipyridamole), Angiotensin-Converting Enzyme (ACE) Inhibitors (e.g., Benazepril (Lotensin), Captopril (Capoten), Enalapril (Vasotec), Fosinopril (Monopril), Lisinopril, (Prinivil, Zestril), Moexipril (Univasc), Perindopril (Aceon), Quinapril (Accupril), Ramipril (Altace), Trandolapril (Mavik); Angiotensin II Receptor Blockers (or inhibitors) (e.g., Candesartan (Atacand), Eprosartan (Teveten), Irbesartan (Avapro), Losartan (Cozaar), Telmisartan (Micardis), and Valsartan (Diovan); Beta Blockers (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren); Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine, (Nimotop), Nisoldipine (Sular), Verapamil (Calan, Isoptin, Verelan); Diuretics (e.g., Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and Spironolactone Aldactone); Vasodialators (e.g., Isosorbide dinitrate (Isordil), Nesiritide (Natrecor), Hydralazine (Apresoline), Nitrates, Minoxidil); Digitalis Preparations (Digoxin and Digitoxin, e.g., Lanoxin); and Statins (e.g., statins, resins, nicotinic acid (niacin), gemfibrozil, clofibrate).
